(19) United States
(12) Patent Application Publication
(10) Pub. No.: US 2018/0334718 A1
(43) Pub. Date: Nov. 22, 2018

(54) Method And System For Detecting Glial Fibrillary Acidic Protein (GFAP), Particularly In Full-term Or Preterm Infants

(71) Applicant: Arne Jensen, Bochum (DE)

(72) Inventor: Arne Jensen, Bochum (DE)

(21) Appl. No.: 15/979,755

(22) Filed: May 15, 2018

(30) Foreign Application Priority Data
May 17, 2017 (EP) 17171487.6

(51) Int. Cl. C12Q 1/6883

(52) U.S. Cl. CPC C12Q 1/6883; C12Q 2600/158

(57) ABSTRACT

The present invention relates to a method for detecting glial fibrillary acidic protein (GFAP) in the blood, in particular in the blood of newborns or preterm infants by means of PCR, that can also be used for detection in the blood of preterm and full-term infants immediately after birth and can be performed so rapidly and reliably that a decision on cord blood therapy for a preterm and full-term infant can be made before severe brain damage occurs. Methods for determining GFAP in the blood of a mammal that are known from the prior art are not usable in preterm and full-term infants as too much blood would be required. The inventive method provides the prerequisite for a therapeutic use of cord blood stem cells to prevent and to therapy infantile cerebral damage that could develop into infantile cerebral paresis. A different possibility does not exist. According to the invention, a method for detecting glial fibrillary acidic protein (GFAP) in the blood of a mammal is provided in which GFAP is determined by means of PCR-amplified immunoassay (I-PCR). The inventive method for detecting glial fibrillary acidic protein (GFAP) in the blood of a mammal by means of I-PCR is preferably combined with other methods to form a system that delivers increased accuracy in detecting oxygen deprivation-induced brain damage, in particular in newborns and preterm infants immediately after birth. These methods consist of determining the head circumference and determining the NO partial pressure in breath gas.

### FIELD OF THE INVENTION

The present invention relates to a method for detecting glial fibrillary acidic protein (GFAP) in the blood, in particular in the blood of newborns or preterm infants.

### BACKGROUND OF THE INVENTION

Glial fibrillary acidic protein (abbrev. GFAP) is a protein that is a main component of intermediate filaments in the cytoplasm of glial cells (in particular astrocytes) of the central nervous system. Its function has not yet been fully elucidated.

Within the central nervous system (CNS), GFAP is predominantly found in astrocytes and can therefore be used with a reasonable level of certainty as a marker for astrocytes. Due to its presence in astrocytes, GFAP plays a significant role as a marker in the diagnostics of brain diseases, such as brain tumors. It is typically found in glial tumors (e.g. astrocytomas, glioblastomas, ependymomas and a number of other glial tumors).

GFAP has also been proposed as a marker for determining concussions. In a concussion, GFAP is released into the blood through the blood-brain barrier. Measurable concentrations are therefore present in the blood up to one week after a concussion (L. Papa et al. “Performance of Glial Fibrillary Acidic Protein in Detecting Traumatic Intracranial Lesions on Computed Tomography in Children and Youth With Mild Head Trauma.” In: Academic emergency medicine: official journal of the Society for Academic Emergency Medicine, Volume 22, No. 11, November 2015, pp. 1274-1282).

GFAP has also been proposed as a marker for determining traumatic brain injuries for similar reasons (T. Bogoslovsky et al. “Fluid Biomarkers of Traumatic Brain Injury and Intended Context of Use”, Diagnostics 2016, 6, 37).

The brain is a complex organ and has a sensitive response to external influences. In contrast to the heart, liver or lung, it does not survive oxygen deprivation for more than eight to ten minutes. In addition, it is particularly sensitive to inflammatory reactions, trauma and genetic aberrations.

The causes of brain damage in newborns and preterm infants are numerous and range from placental circulatory disturbances, clamped vessels during birth, infections, traumatic events during birth and genetic defects to consequences of artificial ventilation in very immature preterm infants. In Germany, around 1000 children suffer very severe brain damage each year as a direct result of oxygen deprivation immediately before, during or after birth. Newborns with a birth weight of lower than 1500 grams are at particular risk. Depending on the extent of the damage and the brain regions affected, they can suffer minor dysfunctions of the brain (e.g. “minimal brain dysfunction”, “attention deficit hyperactivity disorder”) or very severe physical and extensive mental disabilities. These include movement disorders up to arm and leg paralysis, and today even an association with schizophrenia is suspected. The control of emotional impulses can also be impaired, which in adult age can lead to lower mental performance, relationship problems, anxiety, depression or difficulties in adjusting to work.

The burden on those affected and their relatives is considerable and close cooperation between obstetricians, pediatricians, psychologists and speech and movement therapists is needed. The costs that arise for the German collective insurance body are estimated to be at least €500 million for each birth year.

In very preterm infants oxygen deprivation during birth usually damages the white brain matter surrounding the natural hollow spaces (ventricles) inside the brain. As a result of this so-called periventricular leukomalacia, nerves running from the cerebral cortex, inter alia, to the legs are damaged, which causes the mentioned movement disorders. In addition, the sensitive blood vessels of the immature brain can easily tear upon blood pressure fluctuations associated with oxygen deprivation. This results in bleeding into the cerebral fluid or into the white brain matter.

In contrast, in full-term infants, oxygen deprivation leads more to damage in the gray matter of the cerebral cortex, diencephalon and mesencephalon. However, this affects at most 0.04% of infants, while severe brain damage occurs in 10 to 15% of all preterm infants with a birth weight of less than 1500 grams.

Some clinical strategies aimed at protecting infant brain cells and in particular at preventing brain damage in preterm infants have shown good results over the past ten years. Reducing the rate of preterm deliveries is in the foreground, but also early recognition of oxygen deprivation and the appropriate interventions.

Stress situations for the mother and child, bleeding and infections that spread into the uterus increase the risk of preterm delivery. A comprehensive medical history is therefore first gathered in order to recognize risk patients in time.

A comprehensive ultrasound mass screening of infant brains after birth (approximately 5300 children) revealed that all grades of cranial hemorrhages (I to IV) increase with decreasing vitality, in particular in preterm infants (see Berger et al., Eur. J. Obstet. Gynaecol. Reprod. Biol. 75 (1997) 191-203). The so-called Apgar score is used as a measure of vitality, which takes into account children's heart rate, respiratory effort, muscle tone, skin coloration and response to stimulation (0 to 10 points). It is measured at one, five and ten minutes after every birth and is between eight and ten points in healthy children. A low Apgar score may reflect an oxygen deprivation-induced shock state in the child. While lack of oxygen in the brain usually causes the sympathetic nervous system (part of the autonomic nervous system) to increasingly direct blood flow to vital organs from other regions of the organism that are less vulnerable, preterm infants have only a limited capacity for this kind of redirection. This, together with the sensitivity of their blood vessels to blood pressure fluctuations, leads to an extremely low tolerance for oxygen deprivation.

These results led to the concept of early intervention: At the first signs of potential oxygen deprivation, a risk assessment is performed and all preparations made so that the oxygen-deprived infant (decreasing heart rate) is born immediately and under ideal conditions. If successful, an infant in a good state can be further monitored by the pediatrician and damage to the immature brain prevented.

As brain damage cannot always be prevented by screening or early intervention, therapeutic strategies are also being researched to protect and even regenerate the infant brain after damage has already occurred. This requires that the mechanisms leading to brain damage be known.

It was determined that death of oxygen-deprived nerve cells in the brain begins below a threshold value, and then proceeds in two waves: The lack of circulation causes the energy metabolism in the brain to collapse, and in response to the lack of oxygen increased amounts of glutamate are released—one of the most important activating messengers in the brain. If energy is no longer available for ion exchange across the cell membrane, the electrical potential across the membrane of nerve cells can not be maintained either. As a result, large amounts of calcium enter the cells through various ion channels. Some of these channels are regulated by glutamate. The excessive rise in the intracellular calcium concentration, the so-called calcium overload, activates various enzymes that ultimately damage the cells.

Initially, once the acute lack of oxygen is over, the energy metabolism normalizes in several regions of the brain. However, a few hours later a second wave of nerve cell death begins: The nerve cells swell, epileptic activity patterns are measured in the brainwaves, indicating damage to the nerve cells. The reasons for this are thought to include inflammatory reactions and an imbalance between inhibiting and activating messengers in the brain that might trigger programmed cell death (apoptosis). Attempts at intervention are made within this “therapeutic window”, as a significant number of cells are damaged only hours or days following oxygen deprivation.

Various drug-based strategies are already being investigated that are able to alleviate brain damage caused by lack of oxygen, for example, neuroprotective substances, i.e. substances that protect nerve cells. For example, flunarizine is used, which reduces the uncontrolled influx of calcium into oxygen-deprived nerve cells (calcium channel antagonist), and lubeluzole, a glutamate antagonist. Both substances had already shown positive results in stroke. While lubeluzole did not have the desired effect in newborns, flunarizine appears suitable for protecting the infant brain from severe damage resulting from oxygen deprivation.

As any pharmaceutical introduced into obstetrics requires extensive safety evaluations, a search was concurrently made for different substances endogenous to the body that have a similarly beneficial effect. Promising results are already being observed with the energy-rich compound creatine, a metabolic product of the organism (R. Berger et al., Creatine protects the immature brain from hypoxic-ischemic injury, Middelanis et al., 2003, J. Soc. Gynecol. Investig. Vol. 10, No: 2, Abstract No. 290).

Another approach is treatment of brain damage in preterm and full-term infants with cord blood to achieve functional neuroregeneration (A. Jensen, “Stammzellen aus Nabelschnurblut heilen kindlichen Hirnschaden” [Stem cells from cord blood heal infant brain damage] Top Magazine Ruhr, Wissenschaft—Medizin [Science—Medicine] 23(4), 80-81 (2009); A. Jensen, “Erste Therapie eines kindlichen hypoxischen Hirnschadens mit Zerebralparese nach Herzstillstand? [First therapy of hypoxic infant brain damage with cerebral paresis after cardiac arrest?]—Heilversuch durch autologe Nabelschnurstammzell—Transplantation.”[—attempt at healing through autologous cord stem cell transplantation] Regenerative Medizin [Medicine] 4(1), 30-31 (2011)). The publications by A. Jensen et al. “Perinatal brain damage—from neuroprotection to neuro regeneration using cord blood stem cells” Med. Klein. 98(2003) Suppl. 2, pages 22-26 and C. Meier et al. “Spastic paresis after perinatal brain damage in rats is reduced by human cord blood mononuclear cells” Pediatr. Res. 2006; 59(2), pages 244-249 were able to show that systemic transplantation of human mononuclear cells from cord blood into newborn rats with experimental brain damage led both to a massive migration of these cells into the damaged brain region and prevention of spastic pareses. Spasticity, the leading symptom of infant cerebral paresis, was practically no longer detectable in the transplanted rats.

It would, however, be ideal to treat brain-damaged preterm and full-term infants primarily with fresh, and not cryopreserved, autologous cord blood. However for this purpose, it must be determined quickly, still in the delivery room, that treatment is required. The observation of secondary signs, such as a drop in heart rate, is not meaningful enough. However, it was found that even in oxygen deprivation-induced brain damage GFAP is released into the blood through the blood-brain barrier. GFAP can in principle therefore be used as a marker for brain damage in newborns and preterm infants. However, GFAP cannot be determined in the blood of preterm and full-term infants using the method described by L. Papa (see above), because it would require too much blood.

An object of the present invention is therefore to provide a method for detecting glial fibrillary acidic protein (GFAP) that can also be used for detection in the blood of preterm and full-term infants immediately after birth and can be carried out so rapidly and reliably that a decision on cord blood therapy in preterm and full-term infants can be made before severe brain damage occurs.

A further object of the present invention is to provide a method for detecting oxygen deprivation-induced brain damage, particularly in preterm and full-term infants immediately after birth.

### SUMMARY OF THE INVENTION

This object is achieved by a method for detecting glial fibrillary acidic protein (GFAP) in the blood of a mammal, in which GFAP is determined by means of PCR-amplified immunoassay (I-PCR).

I-PCR is described in P. K. Metha et al. “Detection of potential microbial antigens by immuno-PCR (PCR-amplified immunoassay)”, Journal of Medical Microbiology (2014), 63, 627-641, incorporated herein fully by reference. In this method, an antigen to be detected (GFAP according to the present invention) is coupled to an antigen-specific antibody, then optionally to a species-specific biotinylated detection antibody, and via streptavidin to biotinylated DNA to form an antibody-DNA conjugate. The DNA is then amplified by means of PCR.

In principle, a distinction is made between four different I-PCR methods: direct I-PCR, indirect I-PCR, sandwich I-PCR and indirect sandwich I-PCR. Sandwich I-PCR methods and indirect sandwich I-PCR are preferred according to the invention.

According to a first embodiment of the invention, GFAP is detected in the blood of a mammal by means of direct I-PCR, where the blood of the mammal is applied to a microtiter plate followed by the addition of a biotinylated GFAP detection antibody which, after removal of unbound antibody, is further bound to a biotinylated reporter DNA via streptavidin. The DNA is then amplified by means of PCR.

According to a second embodiment of the invention, GFAP is detected in the blood of a mammal by means of indirect I-PCR, where the blood of the mammal is applied to a microtiter plate followed by the addition of a GFAP detection antibody which, after removal of unbound antibody, is further bound to a biotinylated reporter DNA via biotinylated anti-detection antibody and, after removal of unbound antibody, via streptavidin. The DNA is then amplified by means of PCR.

According to a third embodiment of the invention, GFAP is detected in the blood of a mammal by means of sandwich I-PCR, where GFAP capture antibody is immobilized on a microtiter plate, blood of the mammal is added, followed by addition of biotinylated GFAP detection antibody which, after removal of unbound antibody, is further bound to a biotinylated reporter DNA via streptavidin. The DNA is then amplified by means of PCR.

According to a fourth embodiment of the invention, GFAP is detected in the blood of a mammal by means of indirect sandwich I-PCR, where GFAP capture antibody is immobilized on a microtiter plate, blood of the mammal is added, followed by addition of GFAP detection antibody which, after removal of unbound antibody, is further bound to a biotinylated reporter DNA via biotinylated anti-detection antibody and, after removal of unbound antibody, via streptavidin. The DNA is then amplified by means of PCR.

Preferably, GFAP is detected in the blood of a mammal by means of sandwich I-PCR or indirect sandwich I-PCR as this does not require the direct coating of biological samples as source of antigen.

GFAP antibodies are commercially available, for example, from Dianova GmbH, Hamburg, Germany. The GFAP antibody can be added in appropriate dilutions in water, between 1:100 to 1:1000, preferably between 1:300 to 1:700, for example, 1:500.

Preferably, unbound antibodies, unbound streptavidin and unbound DNA are aspirated off and after each step the microtiter plates are washed with a buffer solution, such as TBST buffer.

In the case of indirect I-PCR or indirect sandwich I-PCR, biotinylated goat IgG anti-rabbit, for example, can be used, which is also commercially available, for example, from Dianova GmbH, Hamburg, Germany.

Recombinant streptavidin is also commercially available, for example from Roche, Mannheim, Germany.

According to a first preferred embodiment of the invention, GFAP is detected in the blood of a mammal by means of direct I-PCR, where the blood of the mammal is applied to a microtiter plate, followed by addition of biotinylated GFAP detection antibody which, after removal of unbound antibody, is further bound to streptavidin and, after removal of excess streptavidin, to a biotinylated reporter DNA. After excess DNA is removed, the DNA is then amplified by means of PCR.

According to a second preferred embodiment of the invention, GFAP is detected in the blood of a mammal by means of indirect I-PCR, where the blood of the mammal is applied to a microtiter plate, followed by addition of GFAP detection antibody which, after removal of unbound antibody, is further bound via biotinylated anti-detection antibody and, after removal of unbound antibody, to streptavidin and, after removal of excess streptavidin, to a biotinylated reporter DNA. After excess DNA is removed, the DNA is then amplified by means of PCR.

According to a third preferred embodiment of the invention, GFAP is detected in the blood of a mammal by means of sandwich I-PCR, where GFAP capture antibody is immobilized on a microtiter plate, blood of the mammal is added, followed by addition of biotinylated GFAP detection antibody which, after removal of unbound antibody, is further bound to streptavidin and, after removal of excess streptavidin, to a biotinylated reporter DNA. After excess DNA is removed, the DNA is then amplified by means of PCR.

According to a fourth preferred embodiment of the invention, GFAP is detected in the blood of a mammal by means of indirect sandwich I-PCR, where GFAP capture antibody is immobilized on a microtiter plate, blood of the mammal is added, followed by addition of GFAP detection antibody which, after removal of unbound antibody, is further bound via biotinylated anti-detection antibody and, after removal of unbound antibody, to streptavidin and, after removal of excess streptavidin, to a biotinylated reported DNA. After excess DNA is removed, the DNA is then amplified by means of PCR.

The immunoassay steps described can be automated.

The preferred PCR method according to the invention is real-time PCR. Real-time PCR allows monitoring of the increase in PCR products in real time via fluorescent dyes. Suitable instruments for performing real-time PCR are also commercially available, for example, under the trade names LightCycler (Roche), LightCycler 480II (Roche), Taqman 7900HT (Life Technologies) and ViiA7 (Life Technologies).

The inventive method for detecting glial fibrillary acidic protein (GFAP) in the blood of a mammal by means of I-PCR enables prompt detection of GFAP even with low amounts of blood and is therefore suitable for detection of GFAP in the blood of newborns and preterm infants.

The inventive method for detecting glial fibrillary acidic protein (GFAP) in the blood of a mammal by means of I-PCR is preferably combined with additional methods to form a system that allows increased accuracy in detecting brain damage caused by lack of oxygen, in particular in newborns and preterm infants directly after birth.

According to a further inventive embodiment of the present invention, a combination of the method for detecting glial fibrillary acidic protein (GFAP) in the blood of a mammal by means of PCR-amplified immunoassay is provided together with a method for detecting oxygen deprivation-induced brain damage from breath gas. The breath gas analysis investigates the exhaled breath of a subject. It is known that specific disease markers and metabolites of drugs and metabolic processes can be found in exhaled breath.

It has now been surprisingly found that in oxygen deprivation-induced brain damage nitrogen monoxide NO is formed and is detectable in the patient's breath gas. However, nitrogen oxides are also formed through inflammations in patients' bodies, as described in DE 10 130 296 B4, incorporated herein fully by reference. At first approximation the inflammation-mediated NO concentration in breath gas is constant, while the surge pattern of NO rises abruptly in oxygen deprivation-mediated brain damage.

Therefore, the NO concentration has to be observed relative to a baseline when detecting NO to determine oxygen deprivation-mediated brain damage in a patient's breath gas. To achieve the object, a method for determining nitrogen monoxide in the breath gas of a mammal is therefore provided in which exhaled breath of the mammal is continuously collected and the partial pressure of nitrogen monoxide (NO) continuously measured.

The method for determining nitrogen monoxide in the breath gas of a mammal is preferably performed according to the method described in DE 10 130 296 B4 with the difference that nitrogen monoxide is determined continuously.

For the inventive method for determining nitrogen monoxide in the breath gas of a mammal an apparatus is preferably used that measures the partial pressure of nitrogen monoxide (NO) in the breath gas of a mammal, consisting of a housing with a gas feed, a porous and gas-permeable body that serves as an enrichment element, and a gas-sensitive sensor that generates a signal proportional to the partial NO pressure, where the porous gas-permeable body consists of a carrier material and an absorbent agent, where the absorbent agent is located on the surface of the carrier material and has a selective, reversible absorption capacity for NO. The absorbent agent selected is preferably a calixarene.

The methods are preferably used in combination by first performing a method for determining nitrogen monoxide in the breath gas of a mammal and then using the I-PCR method.

Determining the head circumference of mammals is also suitable to make a further risk assessment and a decision on the use of cord blood therapy. It has been surprisingly found that there is a U-shaped relationship between the head circumferences deviating from the: range between the 25th and 75th percentile and the white matter damage rate (WMD, %), whereas the WMD rate increases, for example, relative to the weight and length percentiles, only at the 10th percentile and below.

According to a further inventive embodiment of the present invention, a combination of the method for detecting glial fibrillary acidic protein (GFAP) in the blood of a mammal by means of PCR-amplified immunoassay is provided together with a method for determining the head circumference of a mammal.

The head circumference can be determined by ultrasound in the womb. Alternatively, or preferably additionally, the head circumference (forehead-back of the head, fronto-occipital) is determined after birth using a simple measuring tape.

According to a further preferred embodiment of the present invention, a combination of the method for detecting glial fibrillary acidic protein (GFAP) in the blood of a mammal by means of PCR-amplified immunoassay is provided together with a method for determining the head circumference of a mammal and with a method for detecting oxygen deprivation-induced brain damage from breath gas.

The object according to the invention is also achieved by a system. for determining brain damage in preterm and full-term infants that comprises an apparatus for detecting glial fibrillary acidic protein (GFAP) in the blood of a mammal by means of PCR-amplified immunoassay (I-PCR) and an apparatus for measuring the partial pressure of nitrogen monoxide in the breath gas of a mammal. Furthermore the system according to the invention preferably also comprises an apparatus for determining the head circumference of a mammal.

The object according to the invention is further achieved by a system for determining brain damage in preterm and full-term infants that comprises an apparatus for detecting glial fibrillary acidic protein (GFAP) in the blood of a mammal by means of PCR-amplified immunoassay (I-PCR) and an apparatus for measuring the head circumference of a mammal.

Preferably, the head circumference of the mammal is determined by cranial ultrasound while still in the womb and then determined after birth using a measuring tape.

Preferably, the use of a database is provided in which the data are collected and the individual risk of the infant is evaluated with respect to whether transplantation (TX) of stem cells from cord blood is indicated.

The inventive system for collecting data allows a hitherto unique ‘real-time’ risk analysis (risks during pregnancy and before, during and after birth) in combination with targeted and timely use of cord blood transplantation to prevent and/or treat infantile brain damage (cerebral pareses).

The inventive system for collecting data on risk factors and findings, including imaging, would further allow to not only document the therapy success but to also define the most effective transplantation time point (which is not yet known event experimentally) and to optimize it individually for the patients in the future, for example, as a ‘learning’ expert system by continuous evaluation of follow-up data.

Storage or interim storage of cord blood at the time of birth is a prerequisite for therapeutic use of cord blood stem cells to prevent and to therapy infantile brain damage that could lead to infantile cerebral paresis. A different possibility does not exist.

The objective of the inventive system is to use the collected data to define a patient group of expecting mothers whose children could benefit from cord blood storage. A further objective of the inventive system is to identify a group of newborns who would benefit from a (short-term) transplantation of fresh or cryopreserved cord blood.

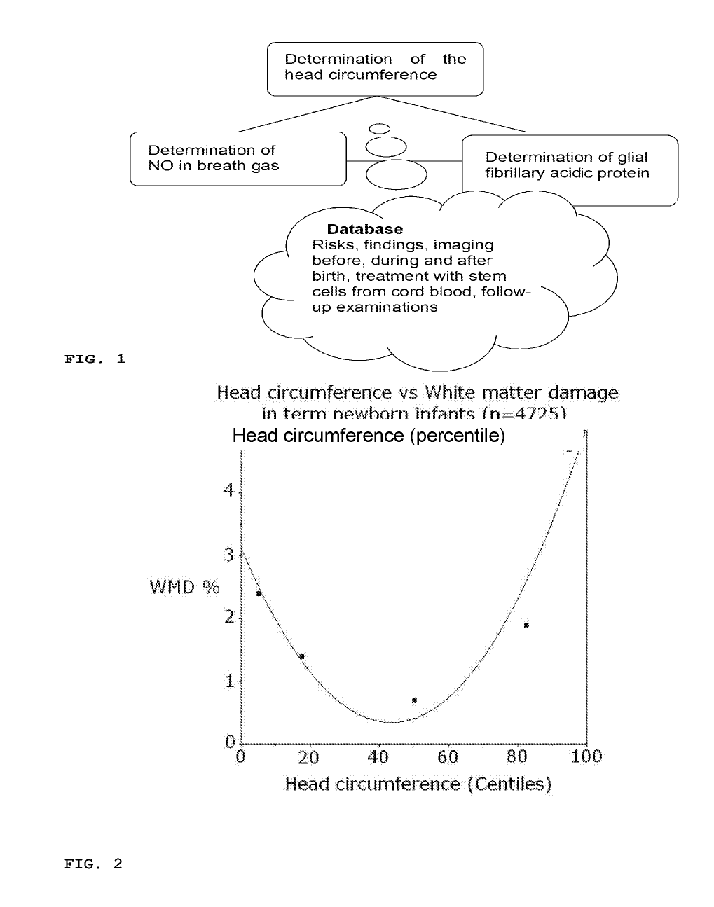

FIG. 1 shows the inventive system for collecting data on head circumference which is preferably determined by ultrasound during pregnancy at regular intervals and stored in a database. The head circumference is preferably also determined after birth using a measuring tape. In addition, measurement data on glial fibrillary acidic protein in the blood of mammals are collected using the inventive system and are preferably fed into a database, together with breath gas analysis data on nitrogen monoxide (NO) if the child is given artificial respiration.

FIG. 2 shows the correlation of the risk of suffering white matter damage (WMD %) relative to the percentiles of head circumference at the time of birth. The brain damage in the white matter (WMD) forms the neuro-anatomical basis for developing cerebral paresis. The data presented as data points determined and as a regression curve relate to 4725 full-term newborns in a prospective brain ultrasound screen (born after 37-43 weeks of gestation, WG) who would usually not be tested by brain ultrasound. The regression curve has the function:

y=3.1168−0.12797*x+0.0014741*x².

The risk assessment with respect to the development of brain damage thus necessitating cord blood storage at the time of birth and potential transplantation of stem cells from cord blood after birth can also be made using other data collected in the database. These include growth retardation of the child, premature contractions, reduced cervical length, macrosomia, especially placental insufficiency, twins/multiple births, age of the mother, bleeding during pregnancy, placental disorders, gestosis/preeclampsia/HELLP syndrome, gestational diabetes, rhesus constellation, changes in CTG, nicotine abuse, alcohol/drug abuse, maternal diseases, infections/fever/cervicitis, feto-fetal transfusion syndrome (FFTS) in multiple births, obesity, hydramnios, breech/transverse lie, anemia, risk of asphyxia, fever during birth, cord complications, bleeding, placenta previa, placental abruption, shoulder dystocia, vaginal breech, premature membrane rupture, operative vaginal delivery (vacuum extraction, forceps delivery), emergency delivery, coagulation disorders, cord blood acidosis (pH), decreased Apgar values after 1, 5, 10 minutes, infant resuscitation, intubation, traumatic birth, immune thrombocytopenia, malformations, diaphragmatic hernia, esophageal atresia, hydrops, hyperbilirubinemia, confirmed brain hemorrhage, confirmed periventricular leukomalacia, neonatal encephalopathy, hydrocephalus, infection, organ failure, multi-organ failure, hypotonia, spasticity, hyperexcitability, Cri du Chat, respiratory failure, hypoxemia, circulatory centralization.

The inventive system preferably comprises a database to collect and process the data, where more preferably additional data collected before, during and/or after birth are also collected in the database. The data collected in the database also allow to make a prognosis on the further psycho-motoric development in preterm and full-term infants such that the collected data allow to predict the level of intelligence (determined e.g. using the Kramer intelligence test, IQ, intelligence quotient), fine motor and coordination skills (determined e.g. using the labyrinth test according to Graf & Hinton), and the neurological development (determined e.g. using the neurological examination according to Touwen) as described in the following examples (1), (2) and (3) where the logistical regression analysis was used to create the formula:

IQ=127.782−1.77*gestational week+0.012+birth weight+2.678+Apgar 10′−6.926* White Matter Damage_present (1)
(R=0.462, F=8.667; p=0.000003)

Labyrinth test=−27.284−5.654*cerebral haemorrhage_present−29.196*green amniotic fluid_present+0.869*gestational week−2.703* White Matter Damage_present (2)
(R=0.508; F=10.986; p=0.000003)

Neurological examination=86.041−9.137*green amniotic fluid present−2.534*presentation+0.003*birth weight−1.902*cerebral hemorrhage_present−4.170*White Matter Damage_present (3)
(R=0.547; F=10.836; p<0.00001).

1. A method for detecting glial fibrillary acidic protein (GFAP) in the blood of a mammal, in which the presence of GFAP is detected by PCR-amplified immunoassay (I-PCR) and the partial pressure of nitrogen monoxide is continuously determined in the breath gas of said mammal.

2. The method as claimed in claim 1, wherein the presence of GFAP is detected by sandwich I-PCR.

3. The method as claimed in claim 1, wherein the presence of GFAP is detected by indirect sandwich I-PCR.

4. The method as claimed in claim 1, wherein the presence of GFAP is detected by indirect I-PCR.

5. The method as claimed in claim 1, wherein the presence of GFAP is detected by direct I-PCR.

6. (canceled)

7. The method as claimed in claim 1, wherein the head circumference of a mammal is determined.

8. A system for determining brain damage in preterm and full-term infants, comprising:
a. an apparatus for detecting glial fibrillary acidic protein (GFAP) in the blood of a mammal by means of PCR-amplified immunoassay (I-PCR) and
b. an apparatus for determining the partial pressure of nitrogen monoxide in the breath gas of a mammal.

9. The system for determining brain damage in preterm and full-term infants as claimed in claim 8, further comprising:
c. an apparatus for determining the head circumference of a mammal.

10. (canceled)

11. The system for determining brain damage in preterm and full-term infants as claimed in claim 8, further comprising a database for collecting and processing data.

12. The system for determining brain damage in preterm and full-term infants as claimed in claim 11, wherein data collected before, during and/or after birth are collected in the database.

13. The system for determining brain damage in preterm and full-term infants as claimed in claim 12, wherein data collected before, during and/or after birth are collected in the database that allow to make a prognosis on the further psycho-motoric development in preterm and full-term infants.
